(19) United States
(12) Patent Application Publication
(10) Pub. No.: US 2012/0283569 A1
(43) Pub. Date: Nov. 8, 2012

(54) SYSTEMS AND METHODS FOR NAVIGATING AND VISUALIZING INTRAVASCULAR ULTRASOUND SEQUENCES

(75) Inventors: Francesco Ciompi, Barcelona (ES); Josepa Mauri Ferre, Barcelona (ES); Oriol Pujol, Barcelona (ES); Xavier Carrillo, Badalona (ES); Petia Radeva, Sant Cugat (ES); Carlo Gatta, Barcelona (ES); Simone Balocco, Badalona (ES); Eduardo Fernandez-Nofrerias, Barcelona (ES); Marina Alberti, Barcelona (ES); Oriol Rodriguez, Tiana (ES)

(73) Assignee: Boston Scientific SciMed, Inc., Maple Grove, MN (US)

(21) Appl. No.: 13/462,733

(22) Filed: May 2, 2012

Related U.S. Application Data
(60) Provisional application No. 61/482,521, filed on May 4, 2011.

(51) Int. Cl. A61B 8/12

(52) U.S. Cl. 600/463

(57) ABSTRACT

A method for processing a sequence of ultrasound frames for display includes receiving a sequence of ultrasound frames; characterizing a morphology of regions within a plurality of the ultrasound frames; determining a frame profile for each of the plurality of ultrasound frames; detecting a plurality of key frames from the plurality of ultrasound frames using the frame profiles of the plurality of ultrasound frames; and displaying at least one of the key frames.

### CROSS-REFERENCE TO RELATED APPLICATIONS

This application claims the benefit under 35 U.S.C. §119(e) of U.S. Provisional Patent Application Ser. No. 61/482,521 filed on May 4, 2011, which is incorporated herein by reference.

### TECHNICAL FIELD

The present invention is directed to the area of imaging systems that are insertable into a patient and methods of making and using the imaging systems. The present invention is also directed to methods and imaging systems for navigating and visualizing intravascular ultrasound sequences.

### BACKGROUND

Ultrasound devices insertable into patients have proven diagnostic capabilities for a variety of diseases and disorders. For example, intravascular ultrasound (“IVUS”) imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety of diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor. There is a need for systems and methods of navigating through, and visualizing, a sequence of IVUS images to allow a practitioner to evaluate the sequence of images and prescribe treatment.

### BRIEF SUMMARY

One embodiment is a method for processing a sequence of ultrasound frames for display. The method includes receiving a sequence of ultrasound frames; characterizing a morphology of regions within a plurality of the ultrasound frames; determining a frame profile for each of the plurality of ultrasound frames; detecting a plurality of key frames from the plurality of ultrasound frames using the frame profiles of the plurality of ultrasound frames; and displaying at least one of the key frames.

Another embodiment is a computer-readable medium having processor-executable instructions for processing a sequence of ultrasound frames. The processor-executable instructions when installed onto a device enable the device to perform actions including receiving a sequence of ultrasound frames; characterizing a morphology of regions within a plurality of the ultrasound frames; determining a frame profile for each of the plurality of ultrasound frames; detecting a plurality of key frames from the plurality of ultrasound frames using the frame profiles of the plurality of ultrasound frames; and displaying at least one of the key frames.

Yet another embodiment is a system for generating and processing a sequence of ultrasound frames. The system includes a catheter and an ultrasound imaging core insertable into the catheter. The ultrasound imaging core includes at least one transducer and is configured and arranged for rotation of at least a portion of the ultrasound imaging core to provide a sequence of ultrasound frames. The system also includes a processor, coupleable to the ultrasound imaging core, for executing processor-readable instructions that enable actions including receiving a sequence of ultrasound frames; characterizing a morphology of regions within a plurality of the ultrasound frames; determining a frame profile for each of the plurality of ultrasound frames; detecting a plurality of key frames from the plurality of ultrasound frames using the frame profiles of the plurality of ultrasound frames; and displaying at least one of the key frames.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an ultrasound imaging system suitable for insertion into a patient, according to the invention;

FIG. 2 is a schematic side view of one embodiment of a catheter suitable for use with the ultrasound imaging system of FIG. 1, according to the invention;

FIG. 3 is a schematic longitudinal cross-sectional view of one embodiment of a distal end of the catheter of FIG. 2 with an imaging core disposed in a lumen defined in a sheath, according to the invention;

FIG. 4 is a schematic block diagram of one embodiment of a method of processing a sequence of ultrasound images, according to the invention;

FIG. 5 is a schematic graph of area of lumen, fibrotic tissue, lipidic tissue and calcified tissue (in order from top to bottom) along a sequence of ultrasound frames, according to the invention;

FIG. 6 is a schematic graph of digitized words for the frames based on morphological characterization; according to the invention;

FIG. 7 is a schematic graph identifying key frames, according to the invention;

FIG. 8 is a schematic display of ultrasound images including the display of multiple key frame images, according to the invention;

FIG. 9 is a schematic display of ultrasound images including the display of multiple key frame images and a bar identifying events in relation to a longitudinal view of the sequence of ultrasound frames, according to the invention; and

FIG. 10 is a schematic display of a longitudinal view of a sequence of ultrasound images with a graph of vessel area and lumen area, a 2.5-dimensional image of the vessel, and a lateral view of the vessel, according to the invention.

### DETAILED DESCRIPTION

The present invention is directed to the area of imaging systems that are insertable into a patient and methods of making and using the imaging systems. The present invention is also directed to methods and imaging systems for navigating and visualizing intravascular ultrasound sequences.

The methods, systems, and devices described herein may be embodied in many different forms and should not be construed as limited to the embodiments set forth herein. Accordingly, the methods, systems, and devices described herein may take the form of an entirely hardware embodiment, an entirely software embodiment or an embodiment combining software and hardware aspects. The methods described herein can be performed using any type of computing device, such as a computer, that includes a processor or any combination of computing devices where each device performs at least part of the process.

Suitable computing devices typically include mass memory and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Nos. 2006/0100522; 2006/0106320; 2006/0173350; 2006/0253028; 2007/0016054; and 2007/0038111; all of which are incorporated herein by reference.

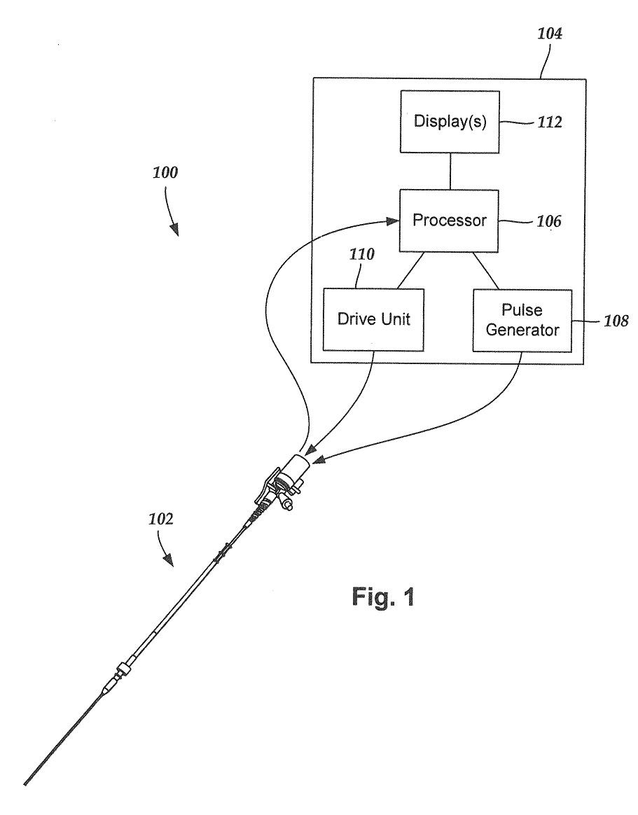

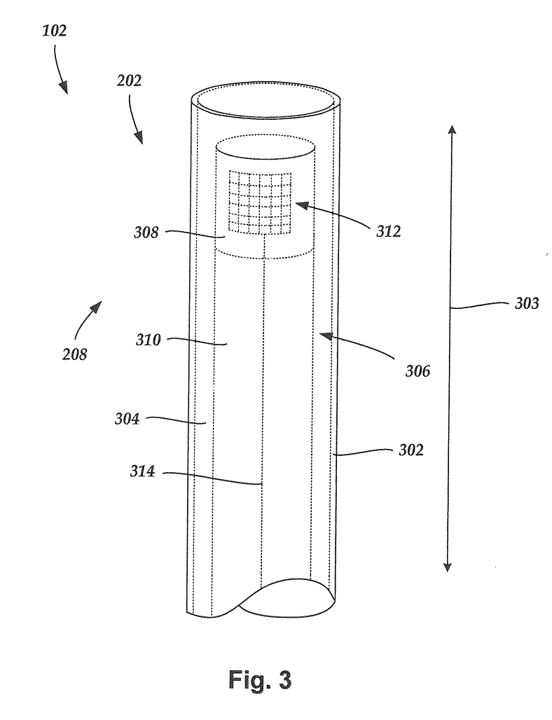

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102.

In at least some embodiments, mechanical energy from the drive unit 110 may be used to drive an imaging core (306 in FIG. 3) disposed in the catheter 102. In at least some embodiments, electric signals transmitted from the one or more transducers (312 in FIG. 3) may be input to the processor 106 for processing. In at least some embodiments, the processed electric signals from the one or more transducers (312 in FIG. 3) can be displayed as one or more images on the one or more displays 112. For example, a scan converter can be used to map scan line samples (e.g., radial scan line samples, or the like) to a two-dimensional Cartesian grid to display the one or more images on the one or more displays 112.

In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

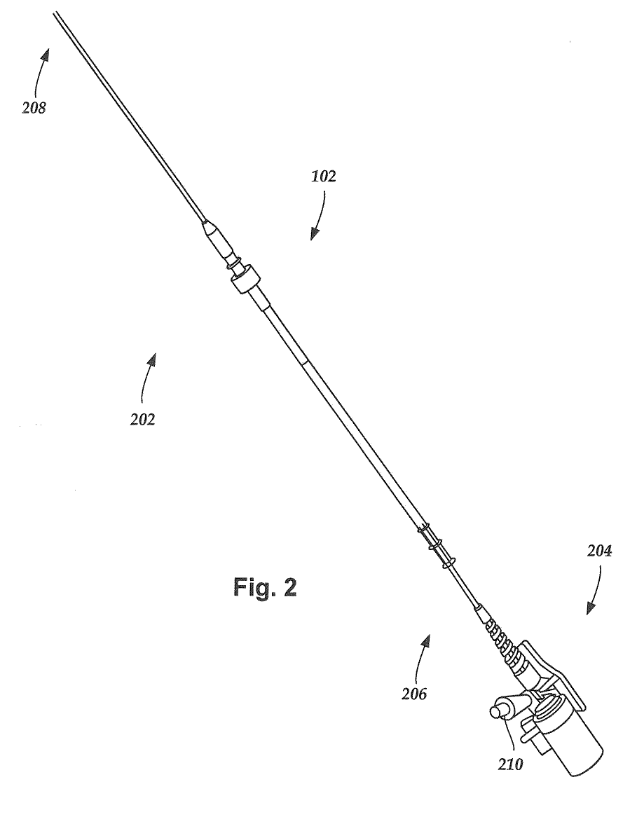

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. Optionally, the catheter 102 may define at least one flush port, such as flush port 210. The flush port 210 may be defined in the hub 204. The hub 204 may be configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 with a longitudinal axis 303 and a lumen 304. An imaging core 306 is disposed in the lumen 304. The imaging core 306 includes an imaging device 308 coupled to a distal end of a driveshaft 310 that is rotatable either manually or using a computer-controlled drive mechanism. One or more transducers 312 may be mounted to the imaging device 308 and employed to transmit and receive acoustic signals. The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In a preferred embodiment (as shown in FIG. 3), an array of transducers 312 are mounted to the imaging device 308. In alternate embodiments, a single transducer may be employed. Any suitable number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 312 are employed, the transducers 312 can be configured into any suitable arrangement including, for example, an annular arrangement, a rectangular arrangement, or the like.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like)

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a matching layer and a conductive backing material formed from an acoustically absorbent material (e.g., an epoxy substrate with tungsten particles). During operation, the piezoelectric layer may be electrically excited to cause the emission of acoustic pulses.

The one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

The imaging core 306 is rotated about the longitudinal axis 303 of the catheter 102. As the imaging core 306 rotates, the one or more transducers 312 emit acoustic signals in different radial directions (i.e., along different radial scan lines). For example, the one or more transducers 312 can emit acoustic signals at regular (or irregular) increments, such as 256 radial scan lines per revolution, or the like. It will be understood that other numbers of radial scan lines can be emitted per revolution, instead.

When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received. In at least some embodiments, the rotation of the imaging core 306 is driven by the drive unit 110 disposed in the control module (104 in FIG. 1). In alternate embodiments, the one or more transducers 312 are fixed in place and do not rotate. In which case, the driveshaft 310 may, instead, rotate a mirror that reflects acoustic signals to and from the fixed one or more transducers 312.

When the one or more transducers 312 are rotated about the longitudinal axis 303 of the catheter 102 emitting acoustic pulses, a plurality of images can be formed that collectively form a radial cross-sectional image (e.g., a tomographic image) of a portion of the region surrounding the one or more transducers 312, such as the walls of a blood vessel of interest and tissue surrounding the blood vessel. The radial cross-sectional image can, optionally, be displayed on one or more displays 112. The at least one of the imaging core 306 can be either manually rotated or rotated using a computer-controlled mechanism.

The imaging core 306 may also move longitudinally along the blood vessel within which the catheter 102 is inserted so that a plurality of cross-sectional images may be formed along a longitudinal length of the blood vessel. During an imaging procedure the one or more transducers 312 may be retracted (i.e., pulled back) along the longitudinal length of the catheter 102. The catheter 102 can include at least one telescoping section that can be retracted during pullback of the one or more transducers 312. In at least some embodiments, the drive unit 110 drives the pullback of the imaging core 306 within the catheter 102. The drive unit 110 pullback distance of the imaging core can be any suitable distance including, for example, at least 5 cm, 10 cm, 15 cm, 20 cm, 25 cm, or more. The entire catheter 102 can be retracted during an imaging procedure either with or without the imaging core 306 moving longitudinally independently of the catheter 102.

A stepper motor may, optionally, be used to pull back the imaging core 306. The stepper motor can pull back the imaging core 306 a short distance and stop long enough for the one or more transducers 306 to capture an image or series of images before pulling back the imaging core 306 another short distance and again capturing another image or series of images, and so on.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 100 MHz.

One or more conductors 314 can electrically couple the transducers 312 to the control module 104 (see e.g., FIG. 1). In which case, the one or more conductors 314 may extend along a longitudinal length of the rotatable driveshaft 310.

The catheter 102 with one or more transducers 312 mounted to the distal end 208 of the imaging core 308 may be inserted percutaneously into a patient via an accessible blood vessel, such as the femoral artery, femoral vein, or jugular vein, at a site remote from the selected portion of the selected region, such as a blood vessel, to be imaged. The catheter 102 may then be advanced through the blood vessels of the patient to the selected imaging site, such as a portion of a selected blood vessel.

An image frame (“frame”) of a composite image can be generated each time one or more acoustic signals are output to surrounding tissue and one or more corresponding echo signals are received by the imager 308 and transmitted to the processor 106. A plurality (e.g., a sequence) of frames may be acquired over time during any type of movement of the imaging device 308. For example, the frames can be acquired during rotation and pullback of the imaging device 308 along the target imaging location. It will be understood that frames may be acquired both with or without rotation and with or without pullback of the imaging device 308. Moreover, it will be understood that frames may be acquired using other types of movement procedures in addition to, or in lieu of, at least one of rotation or pullback of the imaging device 308.

In at least some embodiments, when pullback is performed, the pullback may be at a constant rate, thus providing a tool for potential applications able to compute longitudinal vessel/plaque measurements. In at least some embodiments, the imaging device 308 is pulled back at a constant rate of at least 0.3 mm/s. In at least some embodiments, the imaging device 308 is pulled back at a constant rate of at least 0.4 mm/s. In at least some embodiments, the imaging device 308 is pulled back at a constant rate of at least 0.5 mm/s. In at least some embodiments, the imaging device 308 is pulled back at a constant rate of at least 0.6 mm/s. In at least some embodiments, the imaging device 308 is pulled back at a constant rate of at least 0.7 mm/s In at least some embodiments, the imaging device 308 is pulled back at a constant rate of at least 0.8 mm/s.

In at least some embodiments, the one or more acoustic signals are output to surrounding tissue at constant intervals of time. In at least some embodiments, the one or more corresponding echo signals are received by the imager 308 and transmitted to the processor 106 at constant intervals of time. In at least some embodiments, the resulting frames are generated at constant intervals of time.

In conventional IVUS imaging, two classical views of IVUS images are typically used: a short-axis view, representing a single frame of the sequence, and a long-axis view (or longitudinal view), representing a transversal cut of the three dimensional sequence of frames (e.g., a sequence of frames obtained during pullback of the imaging device). In both views, the content visualization and navigation can often be performed by displacing a cursor along the frame position in the sequence or by changing the angular position in the short-axis view. In this way, a longitudinal view illustrating the desired portion of the vessel can be obtained.

Limitations in currently available visualizations can include, for example; 1) only one frame is visualized in the short-axis view, corresponding to the actual position of the cursor in the long-view which may hinder analyzing the main vessel properties along the sequence; 2) the longitudinal cut offers a view of the vessel morphology limited to the selected angular position in the short-axis, where severe lesions can be invisible due to the choice of angle; and 3) the number of frames available while searching for the best visualization can be very large. Thus, the analysis of the condition of the vessel from a sequence of IVUS frames can involve a time consuming coarse-to-fine manual search performed by, for example, alternately changing the angular position and the longitudinal cursor position.

From a clinical point of view, although the sequence contains a large number of frames, it can often be summarized by substantially fewer frames which display the most important vessel properties. As an example, individual frames of the sequence representing, for example, a stent, a plaque rupture, a lipid pool or a stenosis can be used to characterize a vessel. For this reason, it is would be useful to have direct access to these most salient clinical events, instead of reviewing the whole sequence.

Methods for visualization and navigation along a sequence of IVUS frames (e.g., a sequence obtained during an IVUS pullback procedure) through the most salient vascular phenomena is described herein. This processing task can utilize unsupervised key-frame detection and summarization of IVUS sequences based on the analysis of the morphological vessel profile. The key-frame extraction can be applied to the whole sequence or to any portion or portions of the sequence.

The analysis of changes in the vessel morphology and composition is information that can be used to extract key-frames of IVUS pullbacks. In conventional manual examination of an IVUS sequence, a physician first detects the most interesting frames, i.e., the ones presenting visually and clinically remarkable phenomena. These frames can be manually marked with a label and represent the key frames during the case analysis and review. The marked points define a set of events. Examples of events include stenosis, bifurcations, stent regions, lipid areas, and calcium areas.

When comparing frames of an IVUS sequence for key-frame extraction for IVUS, considerations may include, for example, the continuous (or in some embodiments, repetitive) movement of the probe, together with the heart twisting phenomenon, which may produce continuous local changes in the vessel appearance along the pullback and result in a repeating pattern in the sequences of images. Another possible consideration is the presence of speckle noise produced by scatterers such as blood cells.

In at least some methods of visualizing and navigating the morphological content of a vessel through an IVUS sequence can be automatically detected and visualized. A set of successive frames belonging to the same clinical condition are here defined as an “event”. The automatic determination of events can be performed by an extraction of morphological key frames in the sequence. An automatic classification technique can be applied to group and label frames belonging to homogeneous events. Individual corresponding sections of the vessel can be independently analyzed by selecting the corresponding event.

For example, a key frames detection procedure can include characterization of morphological regions in each frame (or a subset of frames) in the sequence. Morphological profiles are defined with respect to the vessel and these profiles can be discretized and a frame-wise signature can be obtained. Key frames can be selected using a distance criterion applied to the discretized morphological profiles.

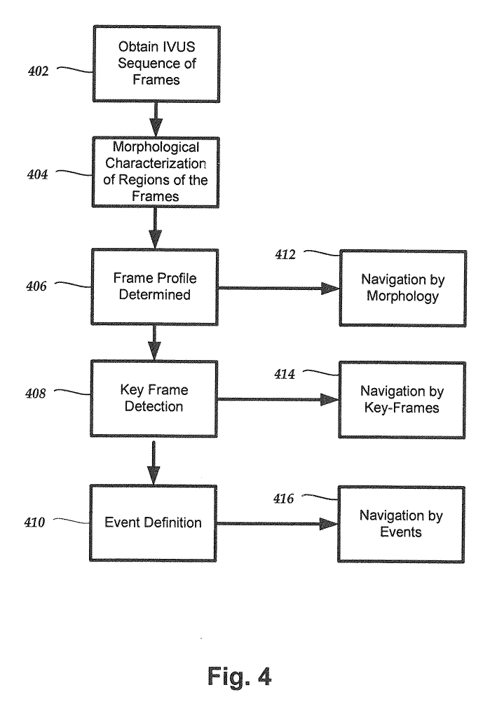

One embodiment of a method for automatic visualization of clinical events and key-frames is depicted in FIG. 4. First, an IVUS sequence of individual frames is obtained (step 402). The morphology of regions in some, or all, of the frames is characterized (step 404). A profile for each of the characterized frames is determined based on the morphological characterization (step 406). Key frames from the sequence are identified by changes in the frame profiles along the sequence (step 408). Events (i.e., a set of successive frames belonging to a same clinical condition) can be identified and defined using the key frames (step 410). This process also allows for navigation by morphology (step 412), navigation by key frames (step 414), and navigation by events (step 416). These steps are described in more detail below.

The IVUS sequence of frames can be obtained (step 402) in any suitable manner including those described above. In at least some embodiments, the acquisition of the IVUS sequence may take into account the effect that the cyclical beating of the heart has on arterial structure. It is known that during the acquisition of IVUS sequences, the heart twisting produces artificial fluctuations of the probe position along the vessel axis (swinging effect). Moreover, due to the heart cyclic contraction/expansion, an apparent rotation with respect to the catheter axis and in-plane translation can often be observed. For these reasons and in order to perform a more reliable analysis of vessel morphology, an optional gating procedure can be used to obtain the IVUS sequence. In some embodiments, frames may be gated based on the cardiac cycle using an electrocardiogram, or the like, to indicate the most stable frames according to the mechanical activity of the heart. In some embodiments, an image-based gating procedure includes performing a motion blur analysis on at least some of the frames. Examples of such gating procedures are described in U.S. patent application Ser. No. 12/898,437 and Gatta, et al., MICCAI 2010, Beijing, LNCS, 2010, Volume 6362/2010, pp. 59-66, both of which are incorporated herein by reference.

Each frame, or at least a subset of the frames of the sequence, is processed to extract a morphological characterization of regions of the frame (step 404). Generally, a set of morphologies is selected for the characterization. In at least some embodiments, interesting morphologies include atherosclerotic plaque, vessel bifurcations, stent, intimal proliferation, thrombosis, and the like. In at least some embodiments, one or more of the following seven different morphologies are defined for characterization: (1) lumen, (2) plaque, (3) adventitia, (4) media, (5) calcium shading, (6) guide-wire effect, and (7) surrounding tissue. It will be understood that only a subset of these morphologies may be used for a particular embodiment and that some embodiments may include additional or alternatively defined morphologies. It will also be understood that one or more of these morphological categories may be each subdivided into two or more separate morphological categories.

Regions of one or more, or even all, of the frames can be classified according to the different defined morphologies. In some embodiment, each region of the frame may be classified. In other embodiments, only some of the regions of the frame are classified. It will be further understood that the size of the regions (e.g., the granularity of the division into regions) can be selected in any manner and the size of the regions may be uniform or non-uniform.

In at least some embodiments, each pixel of the frame (or of selected portions of the frame) is characterized and assigned to one of the defined morphologies. By considering the type and the amount of pixels in each region, clinical measurements can be performed including, for example, lumen area, vessel area, plaque area, and the like. For a given frame, these quantities may be used in a frame profile.

Any suitable classification method can be used. Examples of classification methods are described at U.S. Pat. Nos. 7,460,716; 7,680,307; and 7,778,450 and U.S. patent applications Ser. Nos. 11/285,692; 11/531,133; 12/429,005; 12/253,471; and 12/563,754, all of which are incorporated herein by reference.

In at least some embodiments, the classification of regions of the frame and the computation of the amount of each morphological type for each frame can be performed or refined using a context-based multi-class classification method, such as, for example, a method based on the Error-Correcting Output Codes technique. One example of such a method is designated ECOC-DRF and is described in Ciompi et al., IEEE International Conference on Pattern Recognition, 2010, Istanbul (Turkey), incorporated herein by reference. The conditional probability of a labels field Y={y₁, . . . , y_{K}), where K is the number of defined morphologies, modeled by ECOC-DRF for the regions X={x₁, . . . , x_{M}} of an IVUS frame is:

$$P ( Y \vert X ) = \frac{1}{Z ( X )} {\prod }_{i} {e}^{- {α}_{N} d ( x , {y}_{i} )} {\prod }_{j ∈ {N}_{i}} {e}^{- {α}_{E} d ( {y}_{i} , {y}_{j} , \tilde{x} )}$$

where (i,j) are indexes of nodes in the graph, N, indicates the neighborhood of the node and Z(X) is a normalization function. The graphical representation of the IVUS frame can be a bi-dimensional grid of pixels or groups of pixels, called nodes. The connection between two adjacent nodes/pixels is called an edge. For each node a neighborhood is assumed. The neighborhood can be composed of, for example, the four adjacent pixels around a central pixel or any other configuration disposed around a predefined pixel or node. The exponential terms in the equation above are respectively the node potential, modeling the relationships between a local observation and its corresponding label, and the edge potential, modeling the relationship between labels of connected nodes. Parameters (α_{N},α_{E}) represent the attenuation factors for node and edge potentials. These values can be computed, for example, as explained in Ciompi et al., IEEE International Conference on Pattern Recognition, 2010 Istanbul (Turkey)). The variable d is a distance function defined in the ECOC space (for example, a Euclidean distance) and \tilde{x} is the result of a function of the features for nodes connected by an edge.

The morphological characterization of the regions of the analyzed frames can be used to generate a profile for each of the analyzed frames (step 406). In at least some embodiments, for each IVUS frame, a set of features is computed by combining the output of texture descriptors and of power spectrum analysis. For example, image-based features may be used including, but not limited to, one or more of the following: the response of the image to Gabor filters, Local Binary Patterns, statistical descriptors on gray-level values along sliding windows (mean value and standard deviation), edge detectors, First Order Absolute Moment (FOAM) (as described, for example, in Demi, M.; Comput. Vis. Image Underst., 2005, 97, 180-208, incorporated herein by reference), and the like. Furthermore, features based on frequency domain analysis can also be used, as described, for example, in S. Sathyaranayana, et al., EuroIntervention, 2009, 5, 133-139, incorporated herein by reference.

The feature vector is then assigned to each node of the graph and, for each of the nodes, the probability of belonging to each one of the defined morphologies is computed. In at least some embodiments, through inference, a Maximum A-posteriori Probability (MAP) is obtained for at least some, and possibly all, of the points or regions of the IVUS frame (in polar coordinates). The Maximum A-Posteriori Probability estimation produces, for each frame, a map of labels assigned to each point of the frame according to the defined classifications. Given the label map, the areas of the lumen, vessel, and atherosclerotic plaque can be determined. It will be understood that other known methods for calculating these areas can be used including those that utilize other characterization techniques.

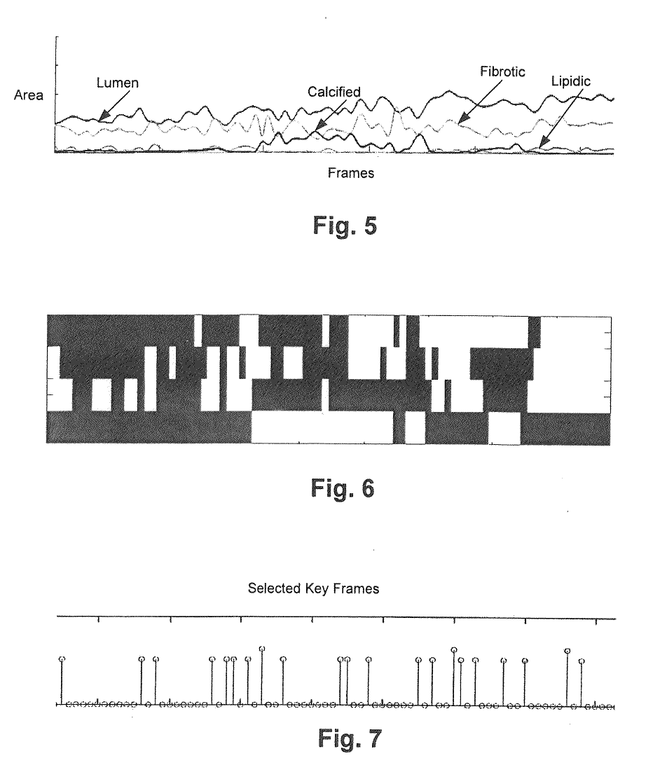

In at least some embodiments, the identified plaque area can be processed by using a plaque characterization technique to add information on the plaque composition. One example of a useful plaque characterization technique can be found in Ciompi, et al., International Journal of Cardiovascular Imaging, 2010, Volume 26, Issue 7, pp. 763-770, incorporated herein by reference. By extending the computation to the whole sequence, a graphical representation of plaque amount along the pullback can be obtained. Further morphological profiles can be added, depicting the amount of additional vessel quantities to achieve a more complete description of the vessel morphology. As an example, in FIG. 5 a set of N=4 profiles is depicted, namely the fibrotic, lipid and calcified plaque profiles, together with the lumen area.

The set of morphological profiles of the vessel can be used as input information for the detection of key-frames (step 408). In at least some embodiments, the profiles can be quantized. In at least some embodiments, the discretization task can be performed by using a fixed size window and taking into account the relationship of local behavior of the signal (inside the window) with respect to the global statistics of the signal itself. As a result, a set of N discretized signals is obtained for the section to be analyzed. From the point of view of each frame in the section, the set of the values for each of the N signals can be grouped Riming an N-symbol codeword. The set of words can be interpreted as a signature of the IVUS sequence, as illustrated, for example, in FIG. 6. The signal discretization can be performed using, for example, the Symbolic Aggregate ApproXimation as described, for example, in Chiu, B., et al, (2003). Probabilistic Discovery of Time Series Motifs. 9^{th} ACM SIGKDD International Conference on Knowledge Discovery and Data Mining. Aug. 24-27, 2003. Washington, D.C., USA, pp 493-498, incorporated herein by reference, and applied to each morphological profile, separately. For instance in FIG. 6, the codewords are composed of four binary symbols. The codeword for the first frame is {0,1,1,0}, where here 0 and 1 correspond to the black and white symbols of FIG. 6 respectively. N may correspond to the number of morphologies characterized or to a subset of those morphologies.

Given a sequence signature, the changes between codewords can be indicative of the vessel local heterogeneity. The detection of key-frames can be achieved by applying a distance measure between codewords. The first frame is considered as the first key-frame. Hamming distance or other metrics can be used as distance measures. In at least some embodiments, the distance measurement is calculated from the numerical label (codeword) of each frame with respect to the preceding key frame.

By defining a minimum distance value, a set of key-frames can be extracted in an unsupervised manner by considering frames that exhibit a distance higher than the fixed threshold with respect to the previous key-frame (see, for example, FIG. 7 in which the key-frames are those indicated by circles above the bottom axis).

The extracted key-frames are ideally representative of significant changes in the vessel morphology. The set of frames comprised between two subsequent key-frames can be considered as belonging to a morphologically homogeneous vessel area.

Given the set of key frames and the vessel signature, the frames of the sequence can be processed and grouped to define clinical events. For this purpose, a supervised learning process can be employed, using the codewords as features and the key frames as markers for the spatial delimitation of the main events.

The processed information allows for at least three different types of visualization/navigation through the IVUS sequence: (1) navigation by key frames (step 412); (2) navigation by events (step 414); and (3) navigation by morphology (step 416).

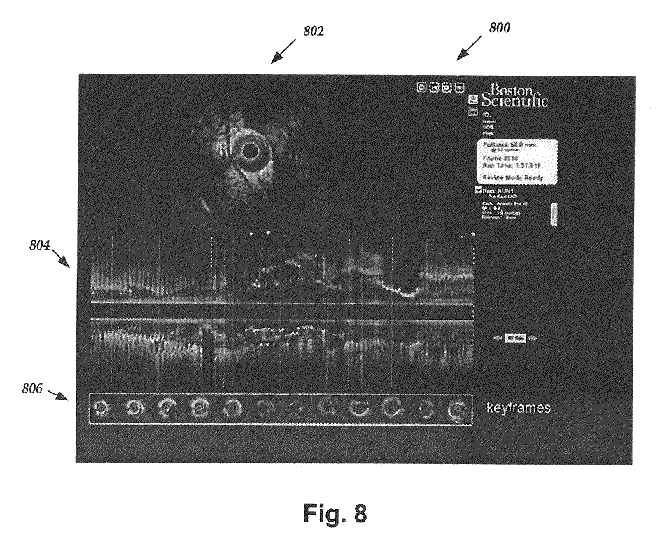

For example, the IVUS sequence can be visualized or navigated by key frames. In this navigation modality, the system first identifies the key frames of the IVUS sequence. Each key frame represents a characteristic of the sequence, and the set of characteristics describes the vessel morphology. In at least some embodiments, the position of each key frame can be marked with a line or other symbol or marker in a longitudinal view of the sequence or a portion of the sequence. In some embodiments, a list of key frames as short-axis views can additionally or alternatively be provided for quickly exploring the content of local changes. FIG. 8 illustrates one example of a display using key frames. The display 800 includes a short-axis view 802 and a longitudinal view 804. The display 800 also includes a series of key frame views 806. In at least some embodiments, the key frame views are selectable and, when selected, the selected key frame view becomes the short-axis view.

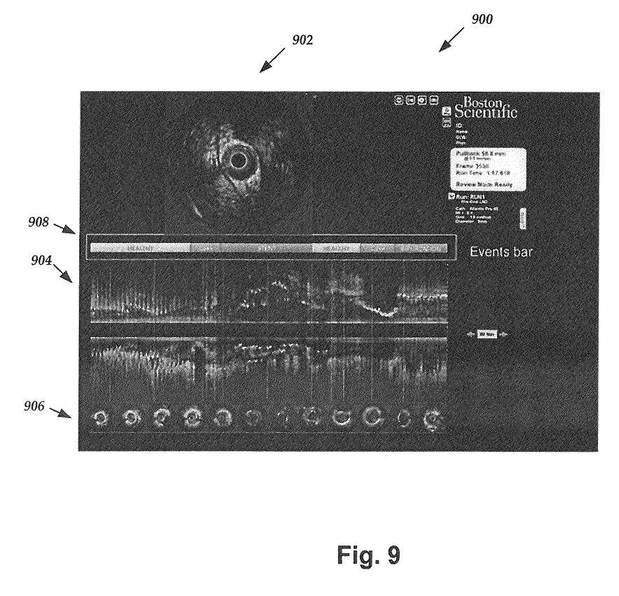

The IVUS sequence can be visualized or navigated by events. Once key frames are identified, the system can identify the main events characterizing the clinical vessel condition. In at least some embodiments, the system is previously trained in a supervised manner in order to associate an event to a set of subsequent keywords in the frame signatures. In this way, the experience learned by physicians is embedded into the system. FIG. 9 illustrates one example of a display using events. The display 900 includes a short-axis view 902 and a longitudinal view 904. The display 900 also optionally includes a series of key frame views 906. In at least some embodiments, the key frame views are selectable and, when selected, the selected key frame view becomes the short-axis view.

In at least some embodiments, the set of detected events can be represented in a bar 908 or other arrangement above or below the longitudinal view 904, and labelled with the name of the corresponding event or some other symbol or marker associated with the event. In some embodiments, each type of event can be differentiated by using a different colour.

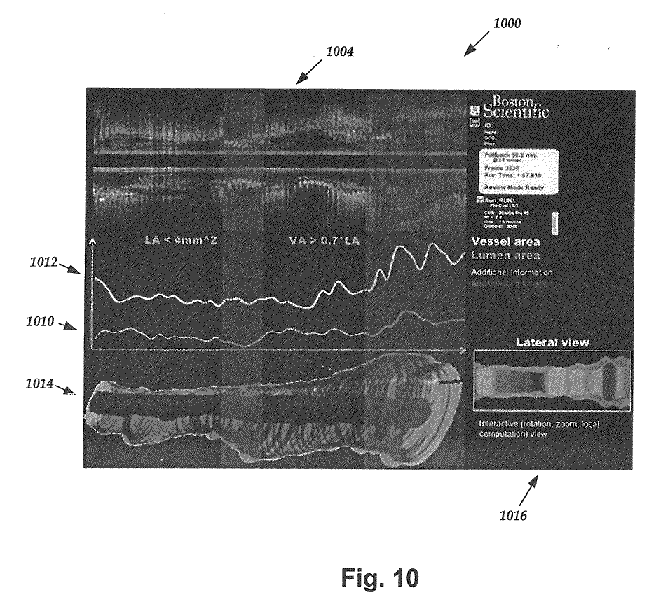

The IVUS sequence can be visualized or navigated by morphological profile. FIG. 10 illustrates one example of a display using morphological profile. The display 1000 includes a longitudinal view 1004.

In at least some embodiments, the characterization of the frames can be used to compute a lumen border or a media-adventia border or both. It will be recognized that other known methods of calculating one or both of these border can also be used. The lumen border, media-adventitia border, or both can be indicated on the short-axis view or longitudinal view. In at least some embodiments, the lumen area 1010, vessel area 1012, or both can be plotted and a 2.5-dimensional representation 1014 of the vessel can be provided.

In at least some embodiments, a lateral view 1016 of the vessel shape can be provided, optionally visualizing with different colours the vessel/lumen narrowing/enlarging or the different morphological regions. The lateral view may be interactive in size, rotation angle, and zoom. Quantitative information on lumen and vessel area at a selected position may be provided. For example, Minimal Lumen Area, Maximal Vessel Area, or both may be provided.

It will be understood that each block of the block diagram illustrations, and combinations of blocks in the block diagram illustrations, as well any portion of the systems and methods disclosed herein, can be implemented by computer program instructions. These program instructions may be provided to a processor to produce a machine, such that the instructions, which execute on the processor, create means for implementing the actions specified in the block diagram block or blocks or described for the systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method for processing a sequence of ultrasound frames for display, the method comprising:
receiving a sequence of ultrasound frames;
characterizing a morphology of regions within a plurality of the ultrasound frames;
determining a frame profile for each of the plurality of ultrasound frames;
detecting a plurality of key frames from the plurality of ultrasound frames using the frame profiles of the plurality of ultrasound frames; and
displaying at least one of the key frames.

2. The method of claim 1, further comprising defining at least one event using the key frames.

3. The method of claim 1, wherein the sequence of ultrasound frames is a sequence of intravascular ultrasound frames.

4. The method of claim 3, further comprising inserting an intravascular ultrasound imaging device into a patient and obtaining the sequence of intravascular ultrasound frames from the intravascular ultrasound imaging device.

5. The method of claim 4, wherein obtaining the sequence of intravascular ultrasound frames comprises obtaining the sequence of intravascular ultrasound frames during a pullback procedure of the intravascular ultrasound imaging device.

6. The method of claim 1, further comprising applying an image-based gating procedure to the sequence of ultrasound frames.

7. The method of claim 1, wherein characterizing the morphology of regions within a plurality of the ultrasound frames comprises, for each region, at least partially characterizing the region by a probability, likelihood, or confidence measure that the region belongs to a one of a set of defined morphologies.

8. The method of claim 1, wherein determining a frame profile for each of the plurality of ultrasound frames comprises generating a numerical label for each of the plurality of ultrasound frames reflecting characterization of the regions of that frame with respect to a plurality of defined morphologies.

9. The method of claim 8, wherein detecting a plurality of key frames comprises determining key frames using a distance measurement calculated from the numerical labels of the plurality of ultrasound frames.

10. The method of claim 9, wherein determining key frames using a distance measurement comprises determining key frames using a distance measurement calculated from the numerical labels of pairs of the plurality of ultrasound frames.

11. The method of claim 1, wherein displaying at least one of the key frames comprises simultaneously displaying a sequence containing a plurality of the key frames.

12. The method of claim 2, wherein defining at least one event using the key frames comprises defining a plurality of events using the key frames, the method further comprising displaying a longitudinal view of the sequence of ultrasound frames and identifying portions of the longitudinal view that correspond to each of a plurality of the defined events.

13. A computer-readable medium having processor-executable instructions for processing a sequence of ultrasound frames, the processor-executable instructions when installed onto a device enable the device to perform actions, comprising:
receiving a sequence of ultrasound frames;
characterizing a morphology of regions within a plurality of the ultrasound frames;
determining a frame profile for each of the plurality of ultrasound frames;
detecting a plurality of key frames from the plurality of ultrasound frames using the frame profiles of the plurality of ultrasound frames; and
displaying at least one of the key frames.

14. A system for generating and processing a sequence of ultrasound frames, comprising:
a catheter;
an ultrasound imaging core insertable into the catheter, the ultrasound imaging core comprising at least one transducer and is configured and arranged for rotation of at least a portion of the ultrasound imaging core to provide a sequence of ultrasound frames;
a processor, coupleable to the ultrasound imaging core, for executing processor-readable instructions that enable actions, including:
receiving a sequence of ultrasound frames;
characterizing a morphology of regions within a plurality of the ultrasound frames;
determining a frame profile for each of the plurality of ultrasound frames;
detecting a plurality of key frames from the plurality of ultrasound frames using the frame profiles of the plurality of ultrasound frames; and
displaying at least one of the key frames.

15. The system of claim 14, wherein the actions further include defining at least one event using the key frames.

16. The system of claim 14, wherein the actions further comprise applying an image-based gating procedure to the sequence of ultrasound frames.

17. The system of claim 14, wherein characterizing the morphology of regions within a plurality of the ultrasound frames comprises, for each region, at least partially characterizing the region by a probability, likelihood, or confidence measure that the region belongs to a one of a set of defined morphologies.

18. The system of claim 14, wherein determining a frame profile for each of the plurality of ultrasound frames comprises generating a numerical label for each of the plurality of ultrasound frames reflecting characterization of the regions of that frame with respect to a plurality of defined morphologies.

19. The system of claim 14, wherein displaying at least one of the key frames comprises simultaneously displaying a sequence containing a plurality of the key frames.

20. The system of claim 15, wherein defining at least one event using the key frames comprises defining a plurality of events using the key frames, the actions further comprising displaying a longitudinal view of the sequence of ultrasound frames and identifying portions of the longitudinal view that correspond to each of a plurality of the defined events.
